(19) United States
(12) Patent Application Publication
(10) Pub. No.: US 2026/0240580 A1
(43) Pub. Date: Aug. 20, 2026

(54) FIRST METATARSAL HALLUX VALGUS CORRECTION USING A LOOPED SUTURE TAPE VIA A SECOND METATARSAL APPROACH

(71) Applicant: TheraMicro Inc., Cordova, TN (US)

(72) Inventors: Kevin E. Varner, Cordova, TN (US); Alan G. Taylor, Cordova, TN (US); Rebecca H. Wahl, Cordova, TN (US)

(21) Appl. No.: 19/542,257

(22) Filed: Feb. 17, 2026

Related U.S. Application Data
(60) Provisional application No. 63/760,820, filed on Feb. 20, 2025.

(51) Int. Cl. A61B 17/82; A61B 17/06; A61B 17/56

(52) U.S. Cl. CPC A61B 17/82; A61B 17/06166; A61B 2017/565

(57) ABSTRACT

Methods for first metatarsal Hallux Valgus correction use a looped suture cord or suture tape via a second metatarsal approach. The suture cord can include a loop on one end and straight (no loop) tails on the opposite end. Such a suture cord can be used to correct bunions in the first metatarsal. The loop of the suture cord is used to lasso the second metatarsal, which eliminates the need for drilled holes therethrough. The suture cord can be passed around the second metatarsal, through the loop, and then through a prepared hole in the first metatarsal. After insertion through the first metatarsal, the suture is tensioned to correct the intermetatarsal angle and held in place with an interference screw, fixation screw, or anchoring mechanism.

### CROSS-REFERENCE TO RELATED APPLICATIONS

This application claims the benefit of priority of U.S. provisional patent application No. 63/760,820, filed Feb. 20, 2025, the contents of which are herein incorporated by reference.

### BACKGROUND OF THE INVENTION

### 1. Field of the Invention

Embodiments of the invention relate generally to the correction of hallux valgus. More particularly, embodiments of the invention relate to apparatus and methods for hallux valgus correction of the first metatarsal using a looped suture tape via a second metatarsal approach.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The first ray of the foot is essentially the big toe. It consists of the first metatarsal, proximal phalanx, and distal phalanx. It is the shortest, strongest, and most important weight-bearing point in the forefoot, carrying 40% of body weight in standing. Hallux Valgus, commonly referred to as a bunion, is a complex valgus deformity of the first ray that can cause medial toe pain and difficulty with shoe wear.

Hallux Valgus is considered one of the most common foot deformities, and is described as “lateral deviation of the hallux and its consequent distancing from the median axis of the body”. Diagnosis is made clinically with presence of a hallux that rests in a valgus and pronated position. Treatment can be nonoperative with shoe modifications for mild and minimally symptomatic cases.

Surgical management is indicated for progressive deformity and difficulty with shoe wear. While there are several surgical options available, there remains a need for improved methods for the treatment of hallux valgus.

### SUMMARY OF THE INVENTION

In summary, embodiments of the present invention provide methods for first metatarsal Hallux Valgus correction using a looped suture tape via a second metatarsal approach. The suture tape is typically less than 60 cm long and less than 15 mm wide with a loop on one end and straight (no loop) on the opposite end. Such a suture tape, as discussed in greater detail below, can be used to correct bunions in the first metatarsal.

The second metatarsal is often too small to accommodate a suture passing hole, therefore a loop is used to lasso the second metatarsal, which eliminates the need for drilled holes. A suture tape with a loop can be used to correct a bunion deformity in the first metatarsal by passing the tape around the second metatarsal, through the loop, and then through a prepared hole in the first metatarsal. After insertion through the first metatarsal, the suture is tensioned and held in place with an interference screw, fixation screw, or anchoring mechanism.

The procedure may be repeated more than once to correct the axis bunion deformity. The looped correction may be used independently or with other bone corrections such as Austin/Chevron or Akin resections.

Embodiments of the present invention relate to a method for the correction of a bone mal-positioning where a bone to be aligned is disposed adjacent a support structure comprising securing a central portion of a suture cord onto the support structure; passing ends of the suture cord through a hole formed in the bone to be aligned; applying tension to move the bone to be aligned into a corrected position; inserting an anchor into the hole to prevent movement of the suture cord relative to the hole; and trimming excess suture cord that extends beyond the anchor in the hole.

Embodiments of the present invention relate to a method for the correction of a bone mal-positioning where a bone to be aligned is disposed adjacent a second bone comprising forming a loop in a central portion of a suture cord, the suture cord having ends extending from the central portion; directing the loop about the second bone; passing the ends of the suture cord through the loop to form a lasso about the second bone; passing ends of the suture cord through a hole formed in the bone to be aligned; applying tension to move the bone to be aligned into a corrected position; inserting an anchor into the hole to prevent movement of the suture cord relative to the hole; and trimming excess suture cord that extends beyond the anchor in the hole.

Embodiments of the present invention relate to a method for hallux valgus correction of the first metatarsal of a patient using a looped suture tape via a second metatarsal approach comprising creating a first incision in the patient adjacent the first metatarsal to provide access to drill the hole therethrough; drilling a hole through the first metatarsal; creating a second incision in the patent adjacent the second metatarsal for providing access to loop the suture cord about the second metatarsal; forming a loop in a central portion of a suture cord, the suture cord having ends extending from the central portion; directing the loop about the second metatarsal; passing the ends of the suture cord through the loop to form a lasso about the second metatarsal; passing ends of the suture cord through a hole formed in the first metatarsal; applying tension to move the first metatarsal into a corrected position; inserting an anchor into the hole to prevent movement of the suture cord relative to the hole; and trimming excess suture cord that extends beyond the anchor in the hole.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates an x-ray of a foot, showing the bones of the foot and the intermetatarsal angle;

FIG. 2A illustrates a preferred suture cord used in the methods according to embodiments of the present invention;

FIG. 2B illustrates a detailed view of the suture cord of FIG. 2A;

FIG. 2C illustrates a suture cord having a non-fixed loop according to embodiments of the present invention;

FIG. 3 illustrates a step of marking the first and second metatarsals of a patient, showing where incisions are to be made;

FIG. 4A illustrates a step of drill a hole of desired diameter (such as a hole drilled using a 3.9 mm drill bit, as illustrated) though the first metatarsal ensuring not to drill into the second (lesser) metatarsal;

FIG. 4B illustrates the hole drilled in the first metatarsal in a skeletal model of the foot;

FIG. 5 illustrates a step of forming an incision, such as a one-inch incision, at the second metatarsal line as marked in FIG. 3, and a step of clearing a passage around and under the second metatarsal to then allow the suture cord (implant) to pass under and around the bone, where both ends of the implant are be outside the initial incision;

FIG. 6 illustrates the step of FIG. 5 performed on a skeletal model, where the suture cord is passed under the second metatarsal, with its loop disposed on a distal side of the second metatarsal, relative to the first metatarsal;

FIG. 7 illustrates the step of passing the “tails” the suture cord through the loop on the opposite end to create a “lasso” effect around the second metatarsal;

FIG. 8 illustrates the lasso formed in the step of FIG. 7 secured snugly about the second metatarsal;

FIG. 9 illustrates the tails of the suture cord being passed through the hole in the first metatarsal using, for example, a flexible passing/hook device;

FIG. 10 illustrates the suture cord being tightened to move the first metatarsal into a corrected position;

FIG. 11 illustrates a step, after proper correction is performed and the desired tension is applied, of locking the construct into position by inserting the proper diameter anchor implant (such as a 3.9 mm anchor, as used in the Figure) into the original hole that was drilled into the first metatarsal;

FIG. 12 illustrates a detailed view of the anchor implant fixed in the hole that was formed in the first metatarsal, noting that the anchor implant is fixed flush or slightly sub-flush with the exterior of the cortical wall;

FIG. 13 illustrates a top view of the full construct, prior to cutting off the ends of the excess suture cord;

FIG. 14 illustrates a suture cord configured to form a fixed loop, according to an exemplary embodiment of the present invention;

FIG. 15 illustrates a suture cord configures to form a non-fixed loop, according to an exemplary embodiment of the present invention; and

FIGS. 16A through 16D illustrate the use of a suture cord, without a loop formed therein, for the correction of hallux valgus, according to an exemplary embodiment of the present invention.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system or technique, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide methods for first metatarsal Hallux Valgus correction using a preferred looped suture tape via a second metatarsal approach. In a preferred embodiment, the suture tape can include a loop on one end and straight (no loop) tails on the opposite end. Such a suture tape can be used to correct bunions in the first metatarsal. The loop of the suture tape is used to lasso the second metatarsal, which eliminates the need for drilled holes therethrough. The suture tape can be passed around the second metatarsal, through the loop, and then through a prepared hole in the first metatarsal. After insertion through the first metatarsal, the suture is tensioned to correct the intermetatarsal angle and held in place with an interference screw, fixation screw, or anchoring mechanism.

Overall, exemplary methods of the present invention involve looping the implant around the second metatarsal followed by “shuttling” the implant through a drilled hole in the first metatarsal, pulling the implant through the hole and shortening the distance to effectively pull the first ray towards the second, closing the intermetatarsal angle to an appropriate angle. Details of methods of the present invention are described in greater detail below.

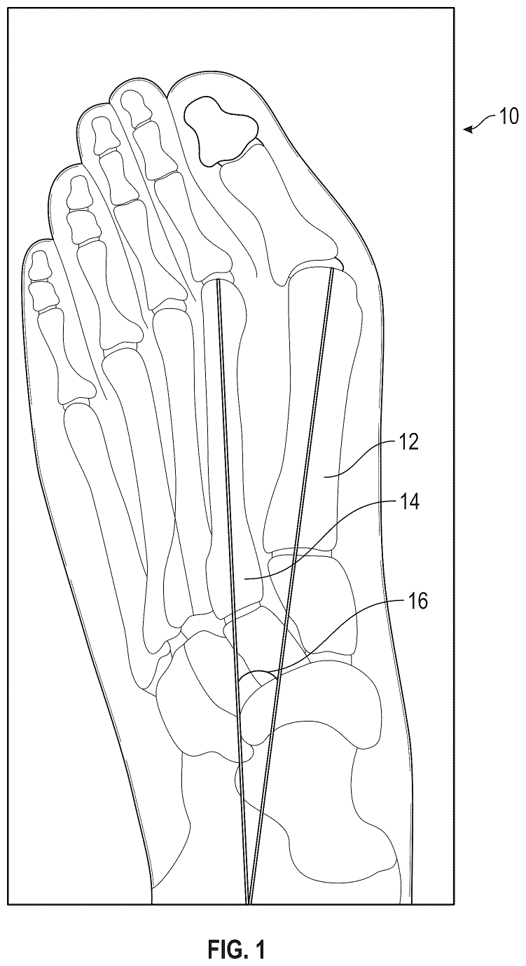

Referring to FIG. 1, an x-ray image of a foot 10 is shown. The foot can include a first metatarsal 12 and a second metatarsal 14. Hallux Valgus provides for an increase in the intermetatarsal angle 16, as shown in the Figure.

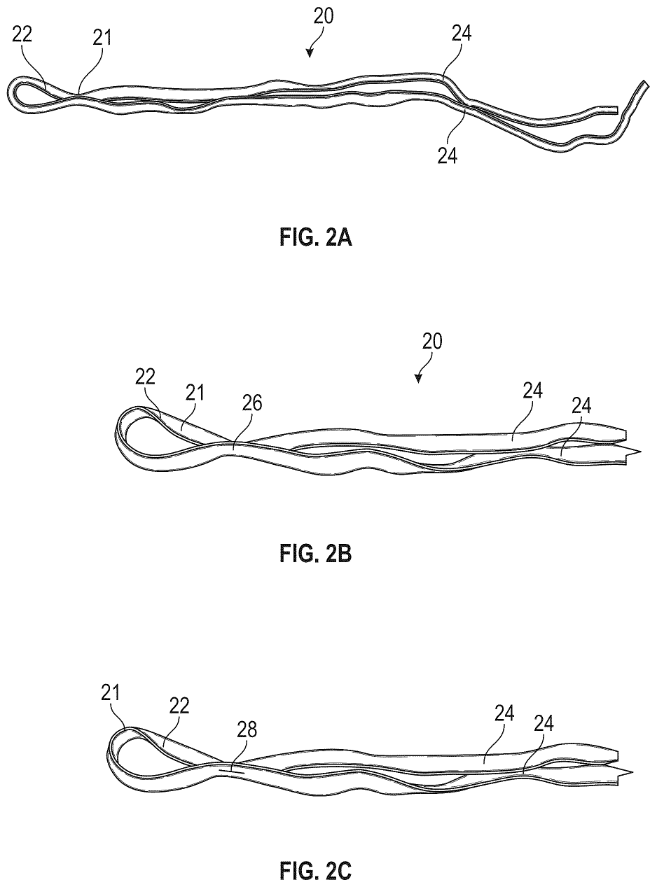

Referring to FIGS. 2A and 2B, a preferred suture cord 20 (also referred to as implant 20 or suture tape 20) comprises a preferred length less than 50 cm and a preferred width less than 15 mm wide. In the preferred embodiment, the suture cord may be composed of polyester, polyethylene, nylon, polyurethane, metallic wire, or the like. The suture cord 20 preferably comprises a central thicker portion 21 and tapering to straight tails 24 extending therefrom. The central portion 21 is preferably planar or flat with a central portion width in the preferred range of 1 mm to 10 mm while the straight tails each comprise a preferably circular profile with a tail width or tail diameter lesser than the central portion width. In the preferred embodiment, the tail width is preferably in the range of 1 mm to 5 mm. Each tail has a preferred length in the range of 10 mm to 200 mm. The central width and tails could both be flat with a preferred width of 1 mm to 15 mm. In the illustrated embodiments shown in FIG. 2, the suture cord 20 may be folded or otherwise configured to form a loop 22 on one end with the pair of straight tails 24 (no loop) disposed on the opposite end. Of course, the size of the suture cord 20 may vary depending on the patient and specific application.

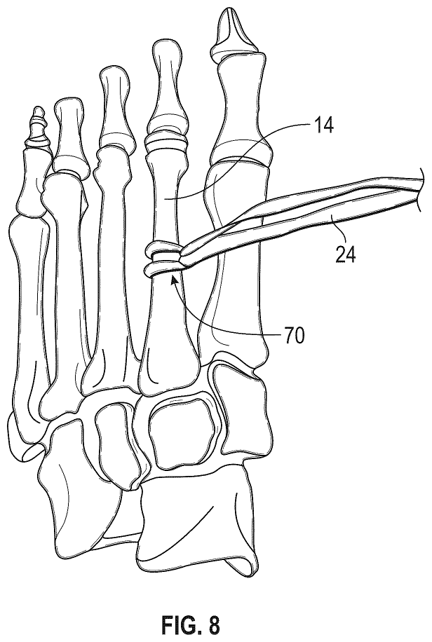

Typically, the loop 22 may be sized so that, when the tails 24 are passed through the loop to lasso the second metatarsal as shown in FIG. 8, the loop connection point 26 (from which the tails 24 extend) is adjacent the second metatarsal. In some embodiments, as shown in FIG. 2C the loop 22 can be formed, for example, by passing one tail 24 through a slit 28 formed along a central portion of the suture cord 20. Thus, the size of the loop 22 may be adjustable by pulling on one of the tails 24. It should be understood that various techniques may be used to make a suture cord with a loop formed centrally therealong and tails extending therefrom.

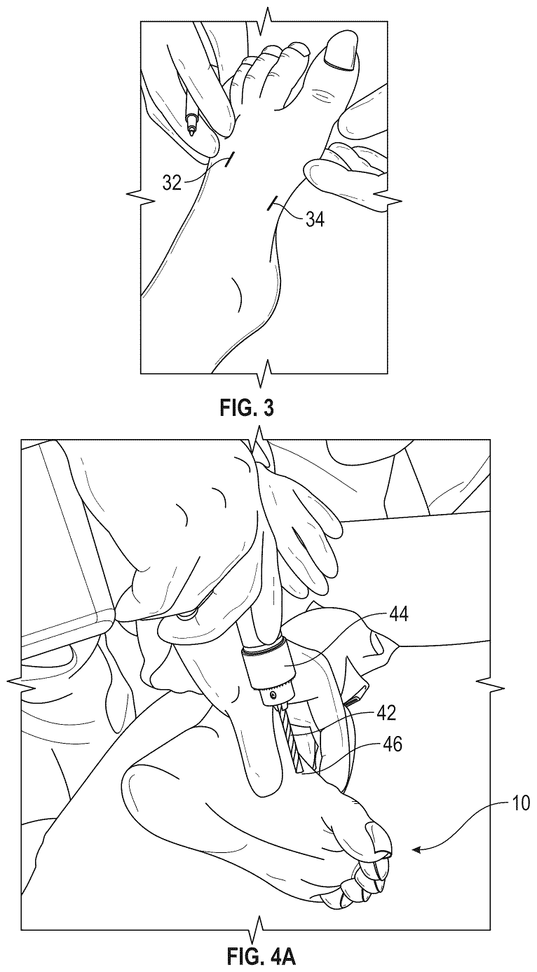

Referring to FIG. 3, at an initial step, the foot may be marked to include a first mark 32, indicating an incision location near the second metatarsal, and a second mark 34, indicating an incision location at the side of the first metatarsal. The incision made at the first mark 32 can be used to lasso the second metatarsal with the suture cord (as described in greater detail below), while the incision made at the first mark 34 can be used to provide access for drilling a hole through the first metatarsal for extending the tails of the lasso therethrough, again, as described in greater detail below. The incisions made at the marks 32, 34 may be, for example, about from about 0.75 to about 2 inches in length, typically about 1 inch in length.

Referring now to FIGS. 4A and 4B, a hole 46 can be drilled through the first metatarsal 12, taking care not to continue to the second metatarsal 14. The diameter of the hole can vary depending on the patient, suture cord size and the like. In FIG. 4A, a 3.9 mm drill bit 42 is driven by a drilling device 44 to form the hole 46. Preferably, the hole 46 is formed laterally through the first metatarsal 12, from one side to the other.

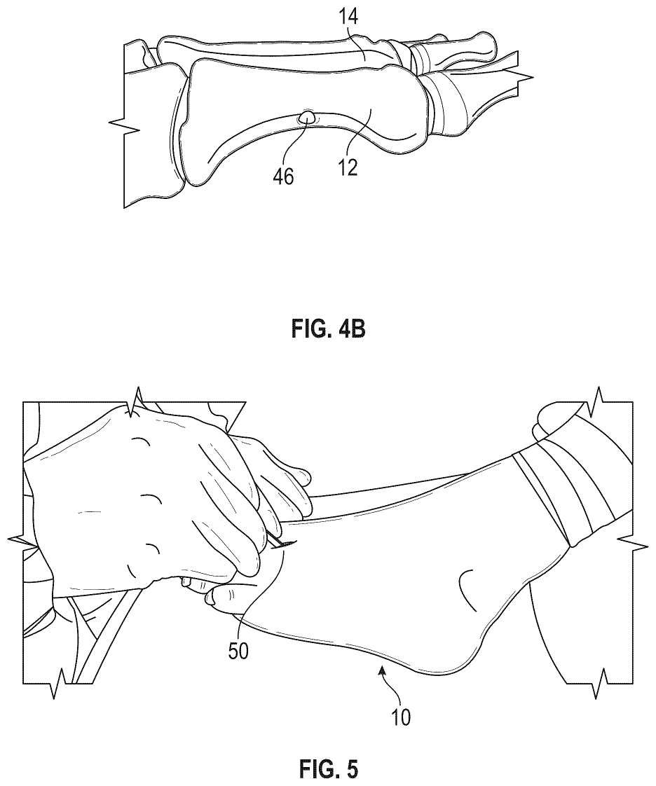

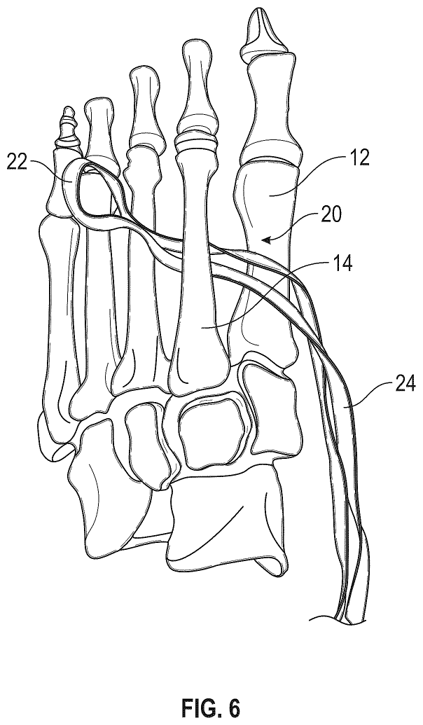

Referring to FIG. 5, an incision 50 can be formed in the foot 10 along the first mark 32 (see FIG. 3), located above the second metatarsal. The incision 50 can be from about 0.75 to about 2 inches in length, typically about one inch in length. The incision 50 provides the surgeon access to the second metatarsal, as described below.

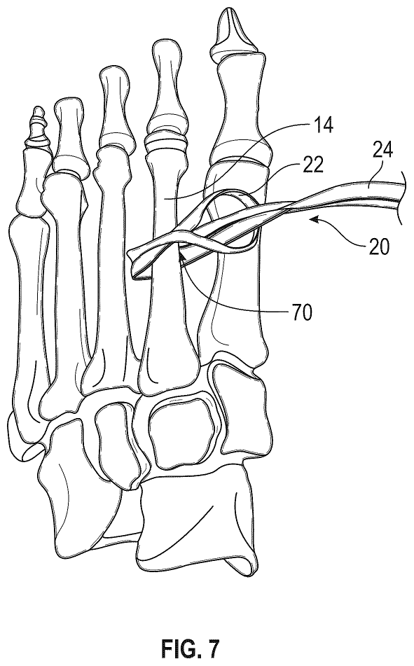

Referring to FIG. 6, once access is provided to the second metatarsal 14 via the incision 50 (see FIG. 5), the surgeon can extend the looped suture cord 20 under the second metatarsal 14, with the loop 22 being distally located, relative to the first metatarsal 12. While this directionality may be reversed, the above described directionality is illustrated in FIG. 6. The loop 22 and the tails 24 can extend out of the incision 50 as the suture cord 20 is positioned around the second metatarsal 14.

Referring to FIG. 7, the tails 24 can be passed through the loop 22, thus forming a lasso 70 for extending completely around the second metatarsal 14 with the suture cord. As shown in FIG. 8, the tails 24 can be pulled to snugly position the formed lasso 70 about the second metatarsal 14.

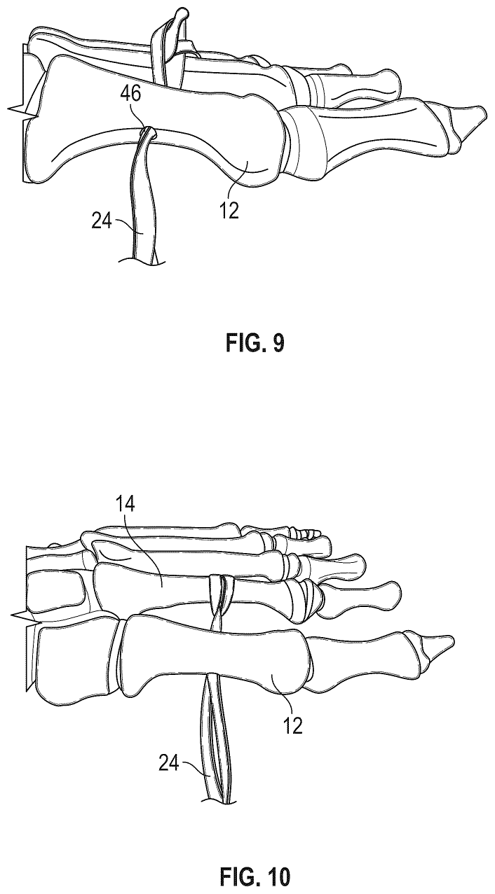

Referring now to FIG. 9, the tails 24 can be maneuvered through the hole 46 that was previously drilled through the first metatarsal 12. Various devices may be used to assist in passing the tails 24 through the hole 46, such as flexible suture passers, hook tools, or the like.

Referring to FIG. 10, the tails 24 can be pulled and the first metatarsal 12 can be positioned to reduce the intermetatarsal angle (see FIG. 1) between the first metatarsal 12 and the second metatarsal 14, thus providing a corrective position for Hallux Valgus.

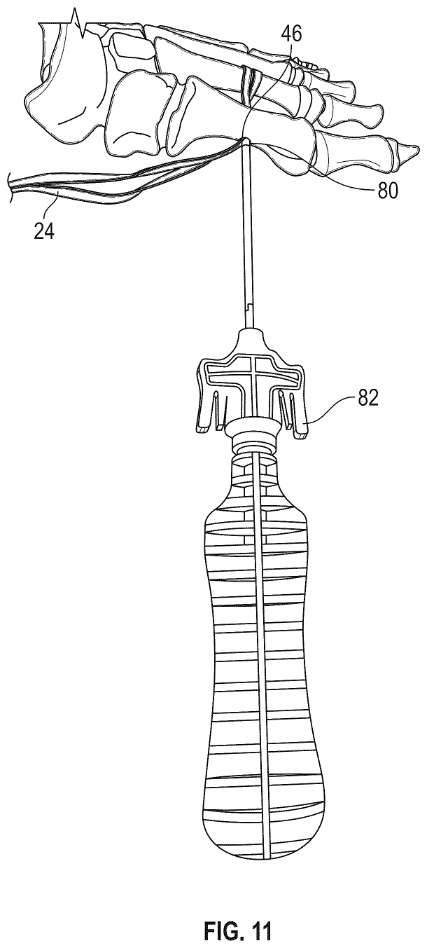

Referring to FIGS. 11 and 12, after proper correction is performed (by the positioning illustrated in FIG. 10) and the desired tension is applied, the construct (suture cord 20) can be locked into position by inserting a proper diameter anchor implant 80, such as a 3.9 mm anchor implant, as used for the 3.9 mm hole in the illustrative embodiment, into the hole 46 that was drilled into the first metatarsal 12. Various anchor implants may be used, such as interference screw, a fixation screw, anchoring mechanism, or the like. A tool 82 may be used by the surgeon for tightening the anchor implant 80 into the hole. As shown in FIG. 12, bi-cortical fixation is performed along with ensuring the proximal end of the anchor implant 80 is flush, or slightly sub-flush, with the exterior of the cortical wall.

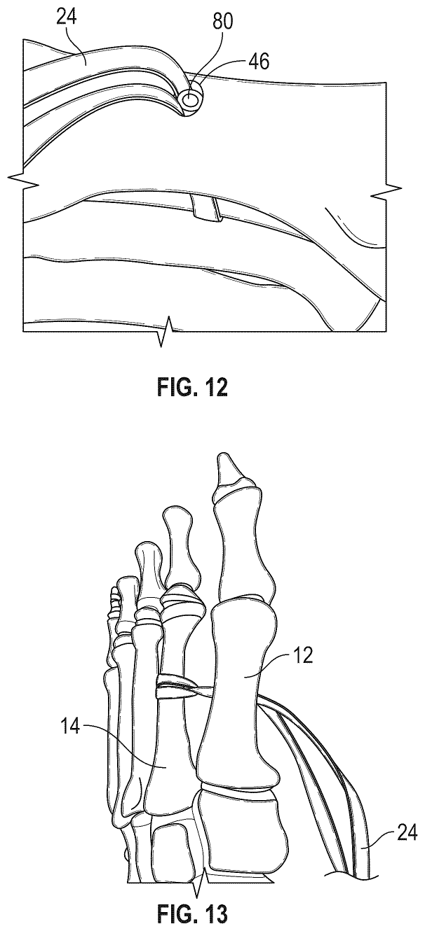

Referring to FIG. 13, the correction can be seen, where the first metatarsal 12 is positioned at a reduced intermetatarsal angle as compared to the angle shown in FIG. 1. The excess tails 24 can be trimmed off and the incisions closed, where the suture cord 20 implant and the anchor implant 80 remains in the patient to continuously provide corrective positioning of the first metatarsal, thus correction the patient's Hallux Valgus condition.

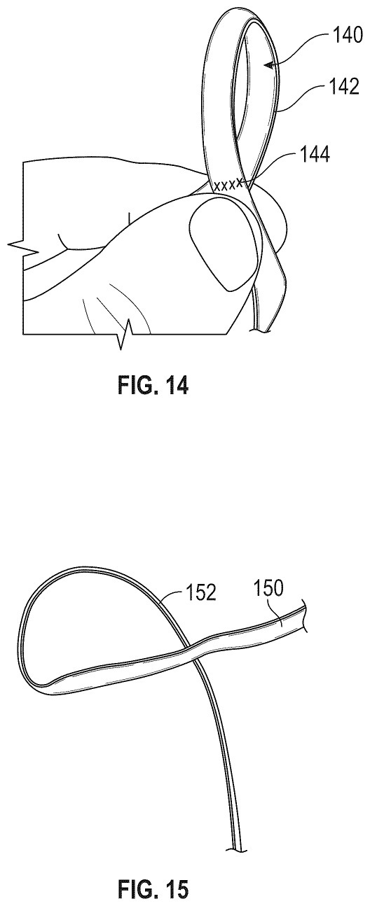

In FIG. 14, a further preferred embodiment of a suture cord 140 comprises a fixed loop 142 that can be formed by overlapping a straight suture tape and affixing it by sewing, adhesive or some others means of forming a loop. FIG. 14 illustrates a stitching 144 that can be used to secure two parts of the suture cord 140 to form the fixed loop 142.

In FIG. 15, a further preferred embodiment of a suture cord apparatus comprises a suture cord 150 configured to form a non-fixed loop 152. The non-fixed loop 152 can be formed by passing one end through the body of the suture implant.

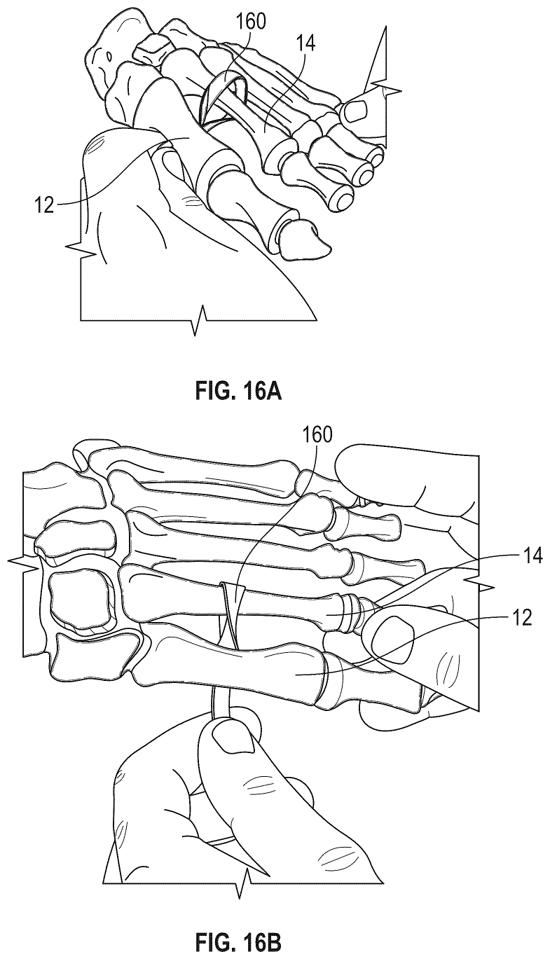

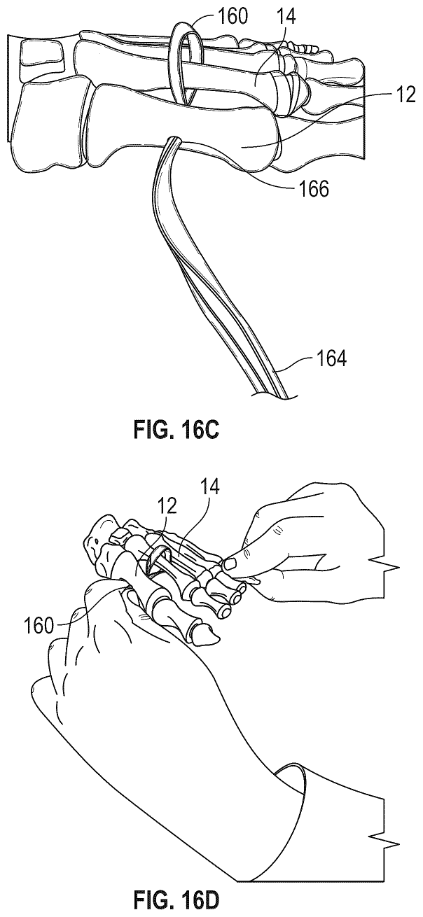

The non-loop suture tape 160 is shown in FIGS. 16A through 16D. This embodiment is similarly passed around the second metatarsal 14, as described above and as shown in FIG. 16A, without passing the ends 164 through a loop feature. The ends 164 are then passed through the first metatarsal drill hole 166 formed in the first metatarsal 12, as shown in FIGS. 16B and 16C The ends 164 are pulled and the first metatarsal 12 can be positioned to reduce the intermetatarsal angle (see FIG. 1) between the first metatarsal 12 and the second metatarsal 14, thus providing a corrective position for Hallux Valgus. An anchor (not shown), as described above, can be used to secure the suture tape 160 in the desired position within the first metatarsal drill hole 166.

While the above discussion focuses on correcting a position of a first metatarsal to correct a Hallux Valgus condition, the apparatus and methods described herein may be used to correct any bone mal-positioning, where the bone may be supported by an adjacent bone or other structure.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A method for the correction of a bone mal-positioning where a bone to be aligned is disposed adjacent a support structure, comprising:
securing a central portion of a suture cord onto the support structure;
passing ends of the suture cord through a hole formed in the bone to be aligned;
applying tension to move the bone to be aligned into a corrected position;
inserting an anchor into the hole to prevent movement of the suture cord relative to the hole; and
trimming excess suture cord that extends beyond the anchor in the hole.

2. The method of claim 1, wherein the suture cord forms a lasso about the support structure to secure the suture cord to the support structure.

3. The method of claim 1, further comprising:
forming a loop in the central portion of the suture cord;
wrapping the loop about the suture structure; and
passing the ends of the suture cord through the loop to form a lasso about the support structure.

4. The method of claim 3, wherein the lasso provides a loop connection point adjacent the support structure.

5. The method of claim 4, wherein the loop connection point is formed by connection sides of the suture cord together.

6. The method of claim 4, wherein the loop connection point is formed by passing one end of the suture cord through a slit formed in a central portion of the suture cord.

7. The method of claim 1, wherein the support structure is a bone adjacent the bone to be adjusted.

8. The method of claim 7, wherein the support structure is a second metatarsal and the bone to be adjusted is a first metatarsal.

9. The method of claim 8, further comprising creating a first incision in a patient adjacent the first metatarsal to provide access to drill the hole therethrough.

10. The method of claim 9, further comprising creating a second incision in the patent adjacent the second metatarsal for providing access to loop the suture cord about the second metatarsal.

11. A method for the correction of a bone mal-positioning where a bone to be aligned is disposed adjacent a second bone, comprising:
forming a loop in a central portion of a suture cord, the suture cord having ends extending from the central portion;
directing the loop about the second bone;
passing the ends of the suture cord through the loop to form a lasso about the second bone;
passing ends of the suture cord through a hole formed in the bone to be aligned;
applying tension to move the bone to be aligned into a corrected position;
inserting an anchor into the hole to prevent movement of the suture cord relative to the hole; and
trimming excess suture cord that extends beyond the anchor in the hole.

12. The method of claim 11, wherein the lasso provides a loop connection point adjacent the support structure.

13. The method of claim 12, wherein the loop connection point is formed by connection sides of the suture cord together.

14. The method of claim 13, wherein the loop connection point is formed by passing one end of the suture cord through a slit formed in a central portion of the suture cord.

15. The method of claim 11, wherein the second bone is a second metatarsal and the bone to be adjusted is a first metatarsal.

16. The method of claim 15, further comprising:
creating a first incision in a patient adjacent the first metatarsal to provide access to drill the hole therethrough; and
creating a second incision in the patent adjacent the second metatarsal for providing access to loop the suture cord about the second metatarsal.

17. A method for hallux valgus correction of the first metatarsal of a patient using a looped suture tape via a second metatarsal approach, the method comprising:
creating a first incision in the patient adjacent the first metatarsal to provide access to drill the hole therethrough;
drilling a hole through the first metatarsal;
creating a second incision in the patent adjacent the second metatarsal for providing access to loop the suture cord about the second metatarsal;
forming a loop in a central portion of a suture cord, the suture cord having ends extending from the central portion;
directing the loop about the second metatarsal;
passing the ends of the suture cord through the loop to form a lasso about the second metatarsal;
passing ends of the suture cord through a hole formed in the first metatarsal;
applying tension to move the first metatarsal into a corrected position;
inserting an anchor into the hole to prevent movement of the suture cord relative to the hole; and
trimming excess suture cord that extends beyond the anchor in the hole.

18. The method of claim 17, wherein the corrected position results in a reduction of an intermetatarsal angle between the first metatarsal and the second metatarsal.

19. The method of claim 17, wherein the lasso provides a loop connection point adjacent the support structure and the loop connection point is formed by connection sides of the suture cord together.

20. The method of claim 17, wherein the lasso provides a loop connection point adjacent the support structure and the loop connection point is formed by passing one end of the suture cord through a slit formed in a central portion of the suture cord.
